(19) United States
(12) Patent Application Publication
(10) Pub. No.: US 2007/0282358 A1
(43) Pub. Date: Dec. 6, 2007

(54) Steerable medical instrument

(76) Inventors: Stan Remiszewski, Spencer, MA (US); Michael E. Abrams, New Haven, CT (US); Dominick L. Mastri, Bridgeport, CT (US); Rich Gambale, Tyngsboro, MA (US); Jeffrey Radziunas, Wallingford, CT (US); Ronald L. Green, Bethel, CT (US); Danial Ferreira, Milford, CT (US)

Correspondence Address: PROSKAUER ROSE LLP, ONE INTERNATIONAL PLACE, BOSTON, MA, 02110, US

(21) Appl. No.: 11/804,871

(22) Filed: May 21, 2007

Related U.S. Application Data
(60) Provisional application No. 60/801,705, filed on May 19, 2006.

(51) Int. Cl. A61B 17/22

(52) U.S. Cl. 606/159

(57) ABSTRACT

A steerable medical instrument includes a control handle, a shaft, steering control wires, and an end effector at the distal end of the shaft. The end effector is a separate component engineered to distribute the stress and strain of bending moments along the length of the end effector to achieve predicable, repeatable, fine motion control over the distal end of the instrument. The end effector may be customized for any medical application. For example, the end effector may comprise a grasping device, a cutting devise, a snare, a specimen retrieval device, or a wound closure device (such as a stapler).

### CROSS-REFERENCE TO RELATED APPLICATION

This application claims the benefit of and priority to U.S. provisional patent application No. 60/801,705 filed on May 19, 2006, which is owned by the assignee of the instant application and the disclosure of which is incorporated herein by reference in its entirety.

### FIELD OF THE INVENTION

This invention relates to the field of multidirectional medical instruments, and more specifically, to steerable surgical instruments.

### BACKGROUND OF THE INVENTION

A number of diagnostic and treatment procedures, once performed surgically through an open wound, are now performed in a less invasive manner with viewing scopes (such as endoscopes and laparoscopes) and catheter instruments. Examples of such instruments include, for example, ERCP cannulas, sphincterotomes (also known as papillotomes), stone balloon catheters and balloon dilatation catheters.

Traditional procedures in which such instruments are utilized include, for example, the removal of stones (such as gallbladder stones), the stretching of narrowed regions in vessels and ducts (strictures), the draining of bile from blocked ducts, or the placement of stents. Some procedures require the use of an electrocautery cutting wire positioned near the distal tip of the instrument. The cutting wire can be used to cut the papilla, intramural duct wall, sphincter, or any other tissue. In many cases, for effective and safe results, the instrument and cutting wire must be precisely located.

In the emerging field of Natural Orifice Transluminal Endoscopic Surgery (NOTES), a viewing scope (e.g., a flexible endoscope) is introduced into a natural orifice in a patient (e.g., the mouth, anus or vagina) and further positioned into a body cavity or other site where surgery is to be performed. A surgical instrument is advanced through a channel of the scope to the desired site. Using NOTES procedures, doctors have removed a woman's gall bladder through the vagina and have performed transgastric appendectomy.

Navigating channels in the human body can be very challenging. Some parts of the human anatomy can be difficult to see and are not always oriented in a convenient location relative to the position of the scope or surgical instrument. Occasionally, the anatomy and the degrees of freedom of the instruments can impede or prevent successful navigation.

A steerable medical instrument is described in US 2003/208219 A1, which is incorporated herein by reference in its entirety. Still, many procedures using steerable instruments remain difficult. A great deal of skill and patience is often required to correctly orient the instrument in a predetermined position.

### SUMMARY OF THE INVENTION

One aspect of the present invention is a steerable medical instrument comprising (i) a shaft comprising a proximal end and a distal end; (ii) an end effector at the distal end of the shaft, said end effector comprising a proximal end and a distal end; (iii) one or more steering control wires anchored in the end effector such that tension applied to the wire proximal to the anchor point causes deflection of the end effector in the direction that tension is applied; and (iv) a control handle connected to the proximal end of the shaft; wherein a material property of the end effector varies along its length to account for variable bending moments experience by the end effector when tension is applied to the one or more steering control wires.

In another aspect, the invention is an end effector for a medical instrument, comprising a flexible member comprising a proximal end and a distal end, wherein the proximal end is attachable to a medical instrument, and wherein a material property of the flexible member varies along its length to account for variable bending moments experienced by the flexible member when the end effector is in use in a patient.

In another aspect, the invention is a method for manufacturing a steerable medical instrument, comprising the steps of forming an end effector comprising a distal end, a proximal end and a longitudinal axis, and creating a plurality of hinge elements disposed along the longitudinal axis. The inventive method may comprise the further steps of anchoring one or more steering control wires in the end effector, and encasing the control wires in a Teflon sleeve to reduce friction. The inventive method may comprise the further steps of providing a shaft having a distal end and a proximal end, and attaching the proximal end of the end effector to the distal end of the shaft; and providing a control handle and attaching the control handle to a proximal end of the shaft.

In another aspect, the invention is a method of positioning a steerable medical instrument in a patient's body, comprising the steps of providing a viewing scope having an instrument channel and an exit port; providing a steerable medical instrument of any of the various embodiments described herein; navigating the scope through the patient's body and positioning the scope near or adjacent a desired area in the patient's body; introducing the steerable medical instrument through the scope and advancing the instrument until the distal end of the instrument protrudes from an exit port of the scope; and steering the distal end of the instrument by tensioning at least one steering control wire.

In yet another aspect, the invention is a method of cannulating the Papilla of Vater in a patient, comprising the steps of providing a flexible endoscope having an instrument channel and an exit port; providing a steerable medical instrument sized to fit through the Papilla of Vater; navigating the endoscope through the patient's body and positioning the endoscope so that its exit port is near or adjacent the Papilla of Vater; introducing the steerable medical instrument through the instrument channel of the endoscope and advancing the instrument until the distal end of the instrument protrudes from the exit port; further advancing and steering the instrument to enter and cannulate the Papilla, wherein the steering is achieved by tensioning at least one steering control wire.

Any of the inventions summarized above (be it an instrument, an end effector, or method) may further comprise one or more of the various features described below, as well as in the detailed description that follows.
(i) The stiffness of the end effector may be varied along its length to account for variable bending moments.
(ii) The end effector comprises a flex tube or beam, whose width may taper down from the proximal end of the effector to the distal end.
(iii) The flex tube or beam comprises one or more hinge elements.
(iv) The hinge elements may be selected from the group consisting of notches and T-bar shaped notches.
(v) The end effector is a composite material.
(vi) The end effector comprises at least one of a grasping device, a cutting device, a snare, a specimen retrieval device, or a wound closure device (such as a stapler).
(vii) A single lumen exits the end effector at a point that is centered on the longitudinal axis of the end effector.
(viii) The shaft contains at least one element having a higher modulus to provide stiffening in the shaft.
(ix) The shaft has a mechanically formed pre-curve section.
(x) The control handle comprises a locking means.

Other aspects and advantages of the invention can become apparent from the following drawings and description, all of which illustrate the principles of the invention, by way of example only.

### BRIEF DESCRIPTION OF THE DRAWINGS

The invention described above may be better understood by referring to the following detailed description taken in conjunction with the accompanying drawings. The drawings are not necessarily to scale, emphasis instead generally being placed upon illustrating the principles of the invention.

FIG. 1 is a drawing of the positioning of an end effector to align with the Papilla of Vater according to an illustrative embodiment.

FIG. 2A is a three-dimensional view of the end effector and the relationship of its cone of motion with the view cone of the endoscope, according to an illustrative embodiment.

FIG. 2B is a schematic drawing of the view cone and cone of motion of the end effector, according to an illustrative embodiment.

FIG. 3A is a drawing of a steerable medical instrument, according to an illustrative embodiment.

FIG. 3B is a blown-up, exploded cross-sectional view of an end effector attached to the flexible shaft of the medical instrument of FIG. 3A, according to an illustrative embodiment.

FIG. 3C is a cross-sectional view of an end effector and a flexible shaft, according to another illustrative embodiment.

FIG. 4A is a cross-sectional view of an end effector having an external cutting wire, according to an illustrative embodiment.

FIG. 4B is a blown-up, cross-sectional view of the junction of the flex tube and the end-effector tip.

FIG. 4C is a blown-up, cross-sectional view of a portion of the flex tube in the end effector of FIG. 4A.

FIG. 4D is an exploded drawing showing the elements utilized in generating an end effector, according to an illustrative embodiment.

FIG. 5A is a cross-sectional view of a flex tube with hinge elements in the form of tapering T-Bar shaped notches.

FIG. 5B is a cross-sectional view of a flex tube with an alternative arrangement of hinge elements.

FIG. 5C is a blown-up drawing of a portion of the flex tube and hinge elements of FIG. 5B.

FIG. 6A is a side-view of an end effector with hinge elements, according to an illustrative embodiment.

FIG. 6B is a three-dimensional view of an end effector with hinge elements, according to an illustrative embodiment.

FIG. 7 is a cross sectional view of a shaft, according to an illustrative embodiment.

FIG. 8A is a drawing of a prior art sphincterotome or papillotome.

FIG. 8B is a drawing of a sphincterotome or papillotome, according to one embodiment of the invention.

FIG. 8C is a drawing of a control handle for a steerable medical instrument, according to an alternative embodiment.

FIG. 8D is a drawing of a control handle for a steerable medical instrument, according to another embodiment.

FIG. 9A is a cross-sectional view of a control handle, according to an illustrative embodiment.

FIG. 9B is a an exploded drawing of the parts of a control handle, according to an illustrative embodiment.

FIG. 10A is a drawing of a three dimensional view of the interior of a control handle comprising a bevel gear with pulley, according to an illustrative embodiment.

FIG. 10B is a drawing of a three-dimensional view of the interior of a control handle comprising a double helix gear, according to an illustrative embodiment.

FIG. 10C is a drawing of a three-dimensional view of the interior of a control handle comprising a double lead screw gear, according to an illustrative embodiment.

FIG. 10D is a drawing of a three-dimensional view of the interior of a control handle comprising a beaded chain gear, according to an illustrative embodiment.

FIG. 10E is a drawing of a three-dimensional view of the interior of a control handle comprising a bevel gear, according to an illustrative embodiment.

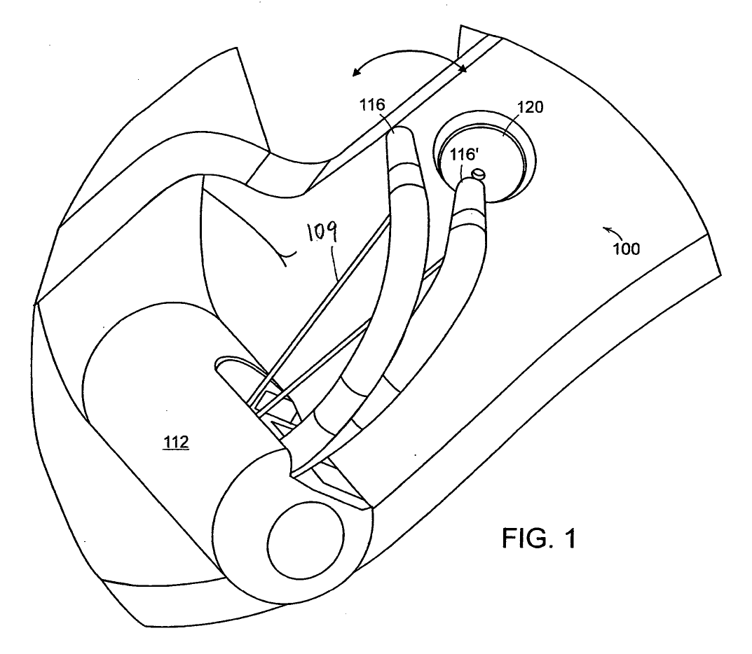

FIG. 1OF is a drawing of a three-dimensional view of a half spur gear in a first position, according to an illustrative embodiment.

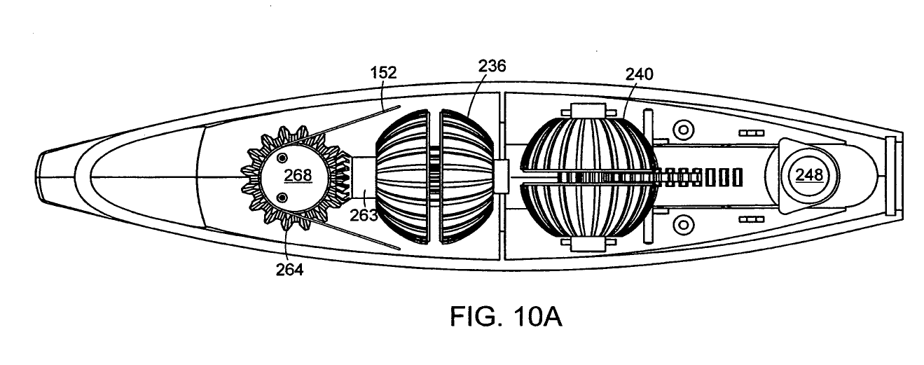

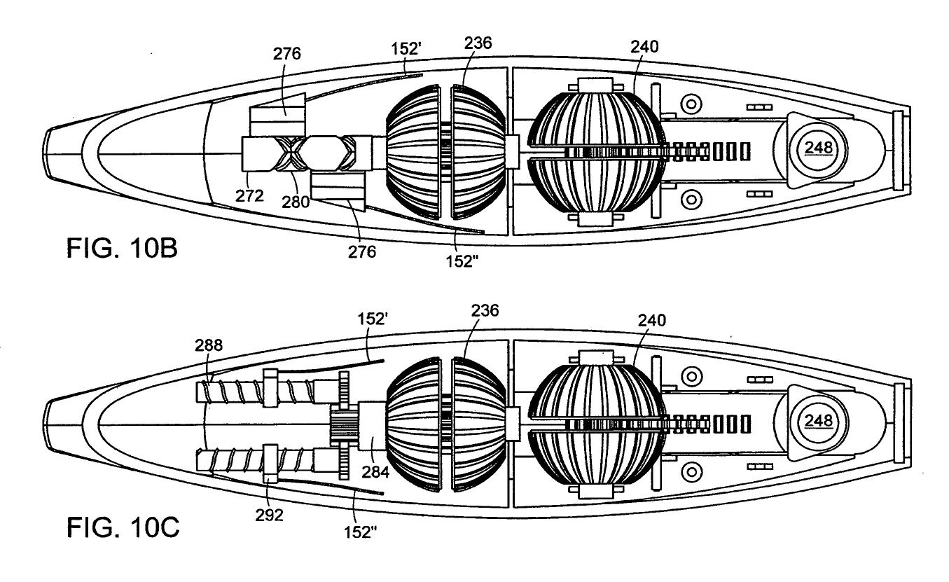

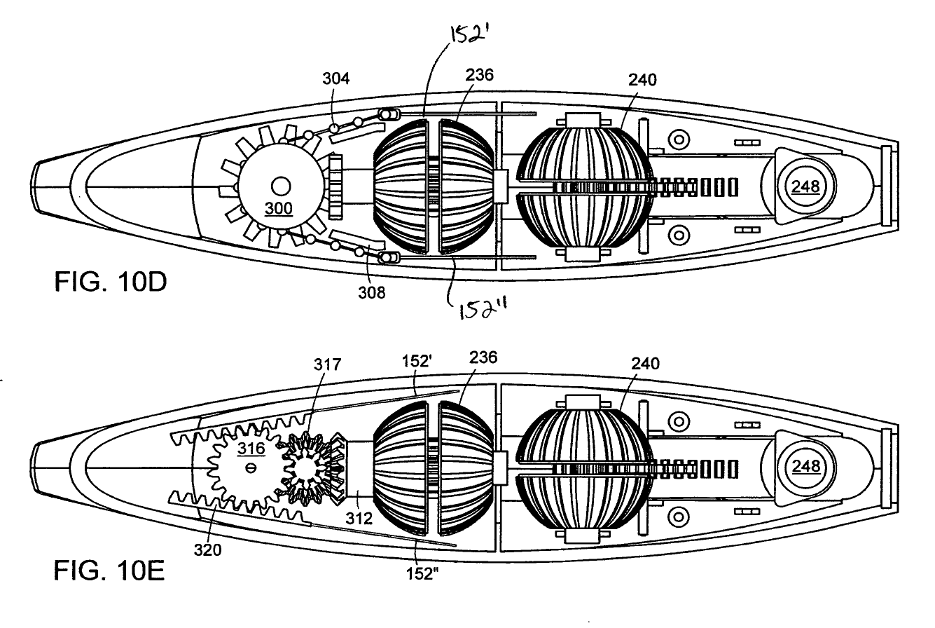

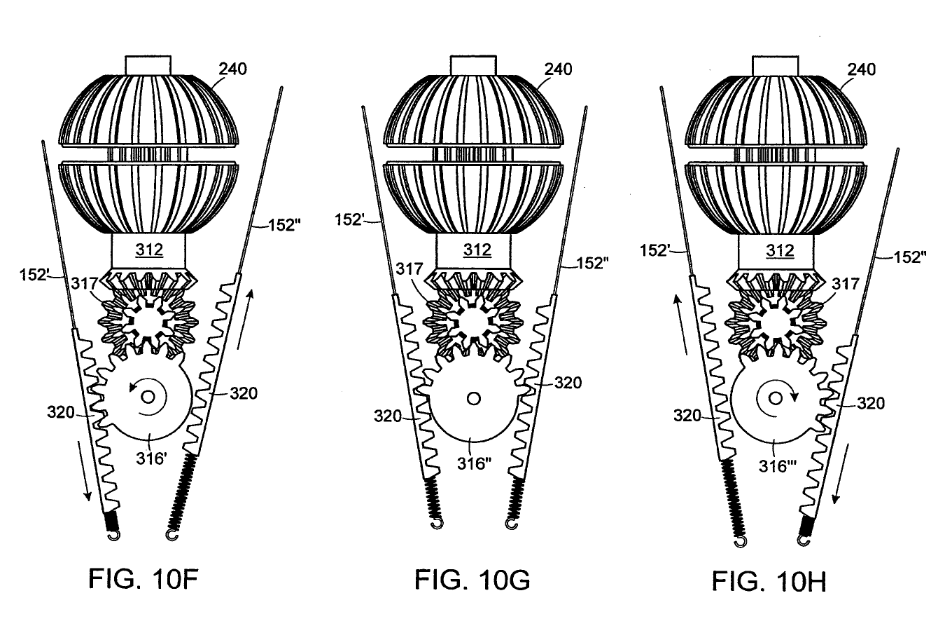

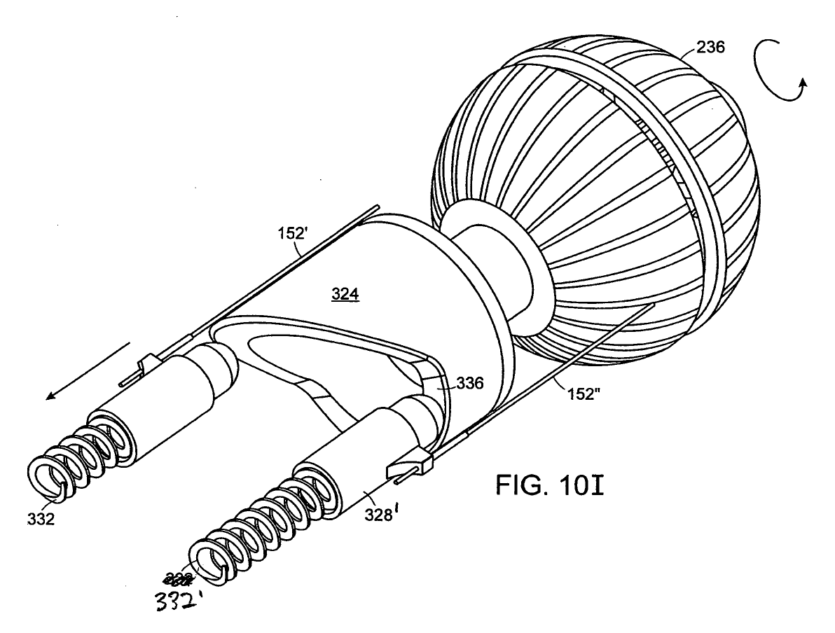

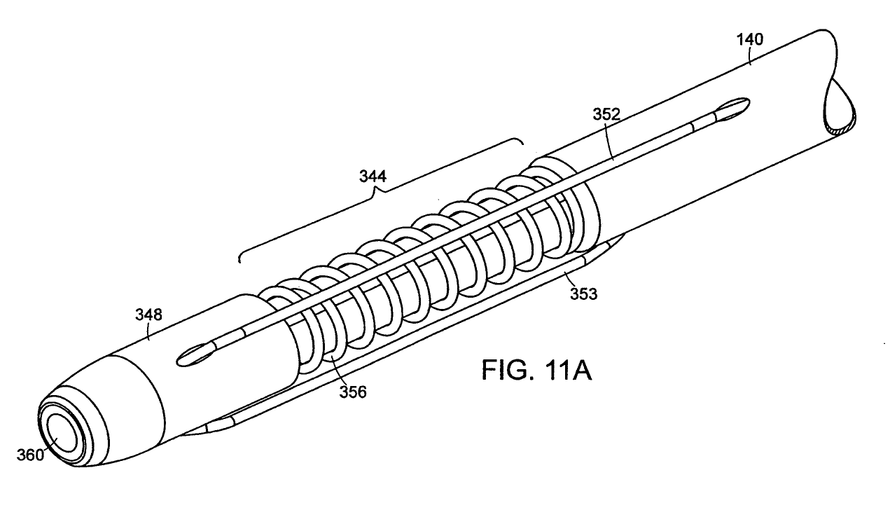

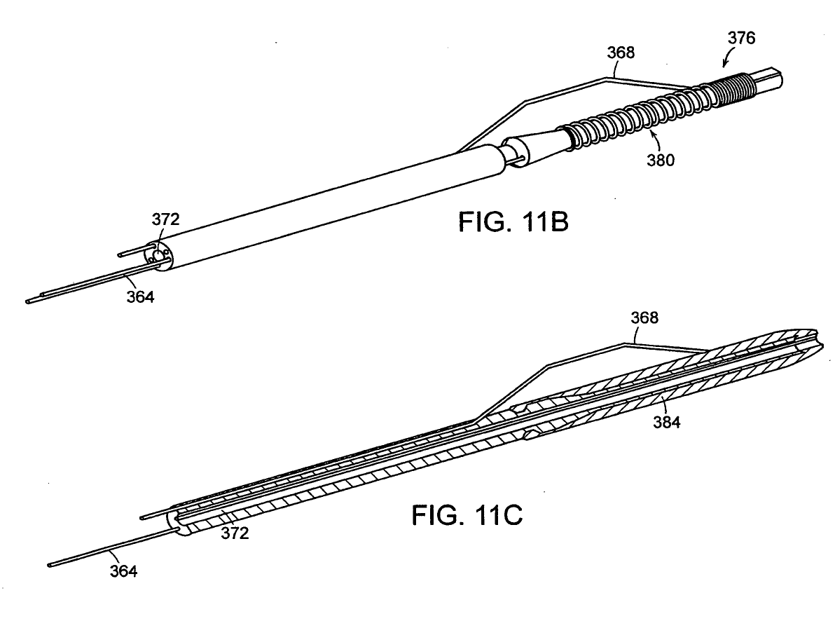

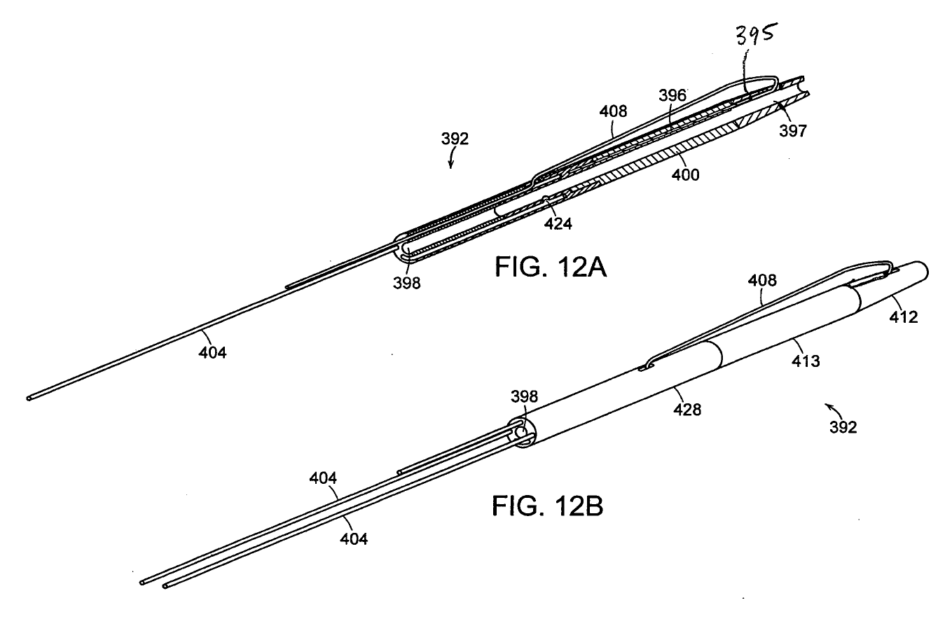

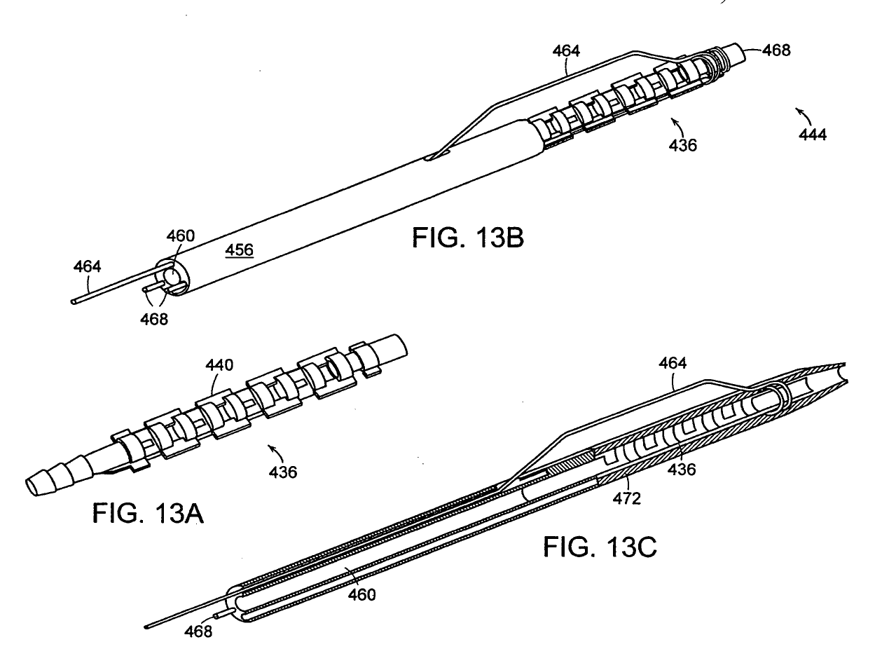

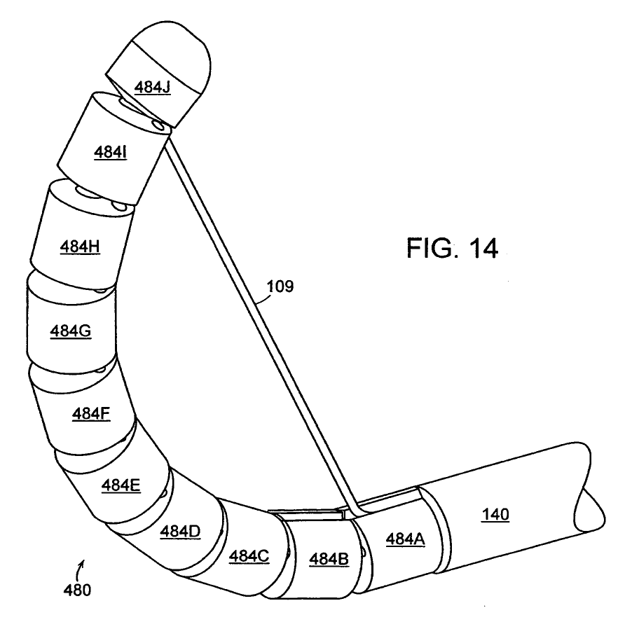

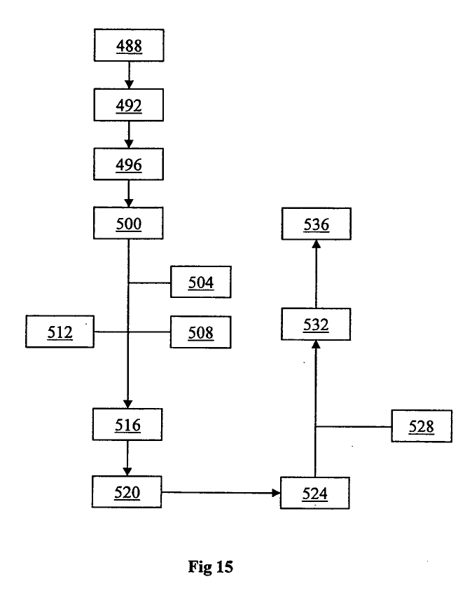

FIG. 10G is a drawing of a three-dimensional view of a half spur gear in a second position, according to an illustrative embodiment.

FIG. 10H is a drawing of a three-dimensional view of a half spur gear in a third position, according to an illustrative embodiment.

FIG. 10I is a drawing of a three-dimensional view of a face cam gear, according to an illustrative embodiment.

FIG. 11A is a drawing of a spring tip end effector, according to an illustrative embodiment.

FIG. 11B is a drawing showing the components of a spring tip end effector, according to an illustrative embodiment.

FIG. 11C is a cross-sectional drawing showing other components of a spring tip end effector, according to an illustrative embodiment.

FIG. 12A is a cross-sectional view of the components of a flex beam end effector, according to an illustrative embodiment.

FIG. 12B is a drawing of additional components of a flex beam end effector, according to an illustrative embodiment.

FIG. 13A is a drawing of a component of a spinal tip end effector, according to an illustrative embodiment.

FIG. 13B is a drawing of other components of a spinal tip end effector, according to an illustrative embodiment.

FIG. 13C is a cross-sectional drawing of the components of a spinal tip end effector, according to an illustrative embodiment.

FIG. 14 is a drawing of a segmented end effector, according to an illustrative embodiment.

FIG. 15 is a flowchart depicting a process for manufacturing a steerable surgical instrument, according to an illustrative embodiment.

### DETAILED DESCRIPTION OF THE INVENTION

FIG. 1 shows the positioning of an end effector 100 of a steerable medical instrument, according to the invention. In this embodiment, the instrument is a sphincterome with a cutting wire 109 in the bowed position, emerging from the distal end of an endoscope 112. The end effector 100 can be moved from a first position 116 to a second position 116′ to orient the end effector 100 to a particular location in the body, such as a papilla 120. In the first position 116, the end effector is in the same plane as the scope. In the second position 116′, the end effector is adjusted out of the plane of the scope in any direction, and in this fashion may be aligned with the axis of the papilla 120. Multi-directional control of the end effector permits the user to control the angle of exit of the end effector from the scope; position the distal tip of an end effector in relation to the patient's anatomy; position a cutting wire (in the case of a sphincterotome) in the correct plane to enable the operator to make a proper cut; and position the end effector 100 yet again in a deeper cannulation. The cutting wire can be made of any conductive material such as stainless steel or a metal-coated fiber.

The steerable medical instrument may be introduced into a patient's body in any number of recognized ways, including, for example, being advanced through the working channel of a viewing scope (for example, an endoscope, colonoscope, bronchoscope, or laparoscope), introduced through a natural orifice in the patient's body (for example, the mouth, outer ear canal, vaginal or anus), or introduced percutaneously.

The end effector may be used in any medical instrument in which it is desirable to have predicable, repeatable, fine motion control over the distal end of the instrument. For example, the end effector may be employed in biliary catheters such as ERCP cannulas, sphincterotomes (papillotomes), stone balloon catheters and balloon dilatation catheters, which can all benefit from multi-directional steering technology. Multi-directional steering technology can reduce biliary procedure time by increasing the number of degrees of freedom of motion of endoscopic instruments, decreasing the occurrence of irritation of the papilla and surrounding areas, and reducing the number of devices and device exchanges required during an endoscopic procedure. Multi-directional biliary catheters employing the end effectors of this invention provide users with fine motion (device) control, in contrast to gross motion (scope) control.

The end effector of this invention may be a component separate from the other components of the instrument, often serving a different purpose than the other instrument components, and may be customized for a particular medical procedure. For example, the end effector may be a separate component fixedly attached to medical instrument, removably attached to the instrument, or in some cases the end effector may be integral with the shaft of the instrument. In addition, the end effector may comprise any number of recognized medical or surgical tools, such as a grasping device, a cutting device, a snare, a specimen retrieval device, or a wound closure device (e.g. a stapler).

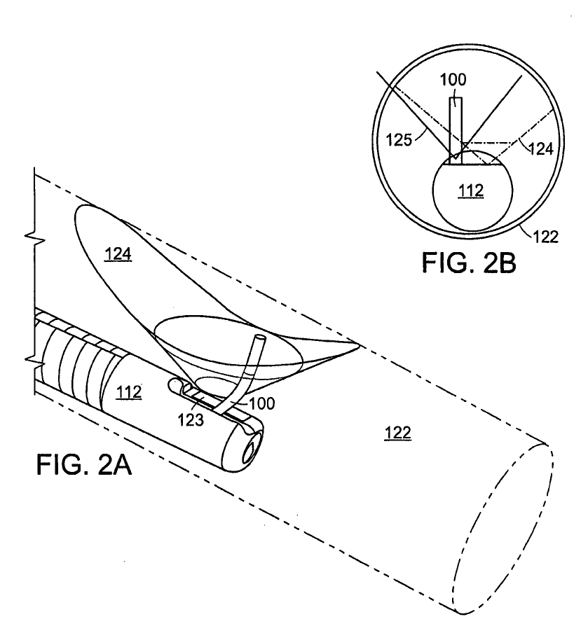

FIG. 2A demonstrates the end effector 100 emerging from the distal end of an endoscope 112 navigating through a patient's anatomy 122. The distal end of the medical device has a lens 123 that can provide the user with a view cone 124 of the patient's anatomy. The view cone 124 includes all of the points that a user may view through the lens 123 of the endoscope 112. In some instances, the view cone 124 may be truncated by the patient's anatomy.

FIG. 2B demonstrates the interaction between the view cone 124 of the endoscope and the cone of motion 125 of the instrument. While the view cone 124 demonstrates the area visible to the user, the end effector 100 can be articulated to be placed anywhere within the cone of motion 125. In some embodiments, the end effector is limited to movement in area overlapping the view cone 124 and the cone of motion 125.

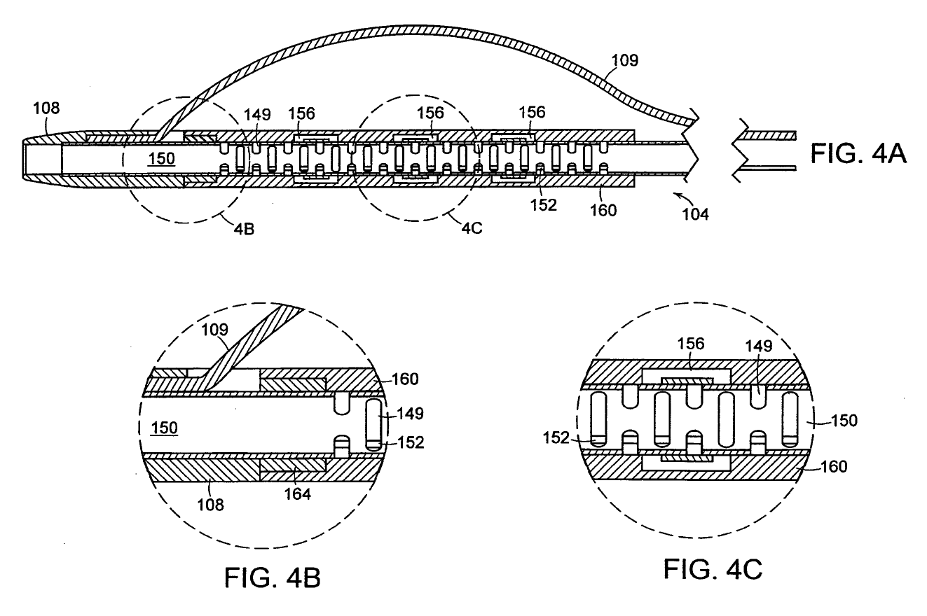

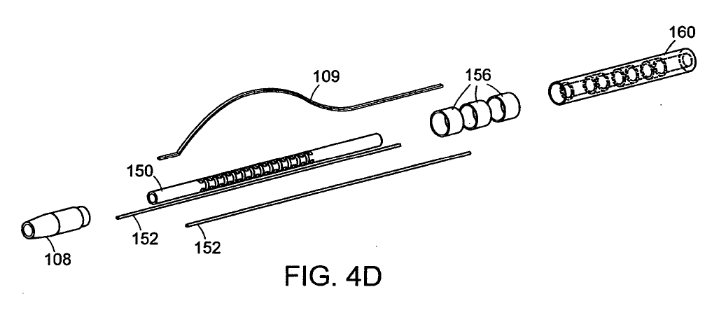

In general, the end effectors of applicants' invention are steered by applying a bending moment to the end effector at any place along its longitudinal length, preferably near the distal tip. In a preferred embodiment, the end effector has one or more steering control wires anchored to or within it. When a tensioning force is applied to a control wire, the end effector will flex or bend in the direction of the tensioning force. Steering control wires 152 are illustrated in FIGS. 4A-4D. The steering control wires may be substituted with recognized equivalents such as high modulus polymer filaments or carbon fiber. The steering control wires can be made of metals commonly used in medical devices, such as stainless steel.

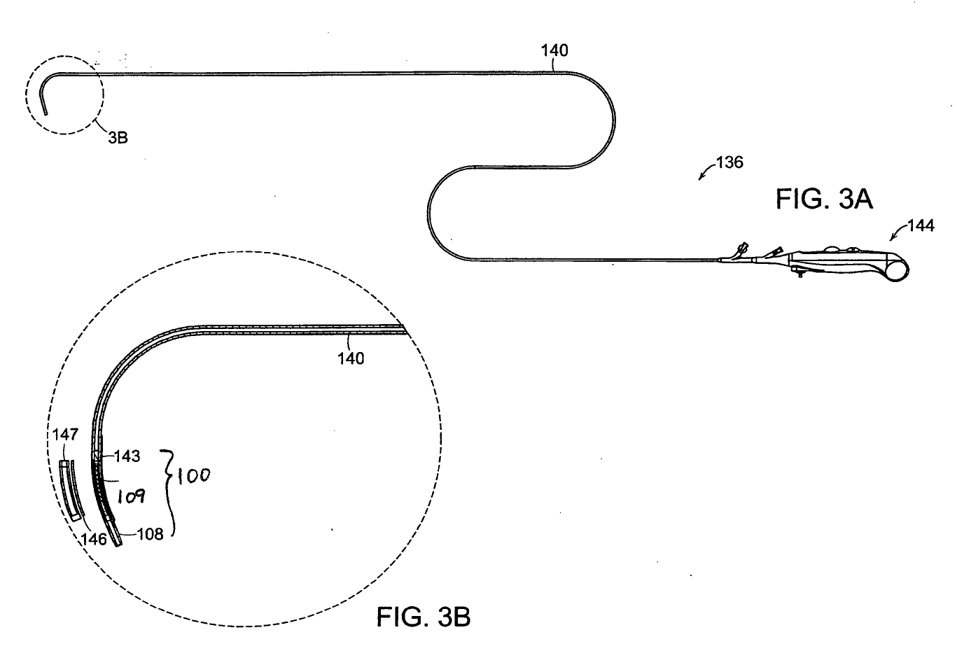

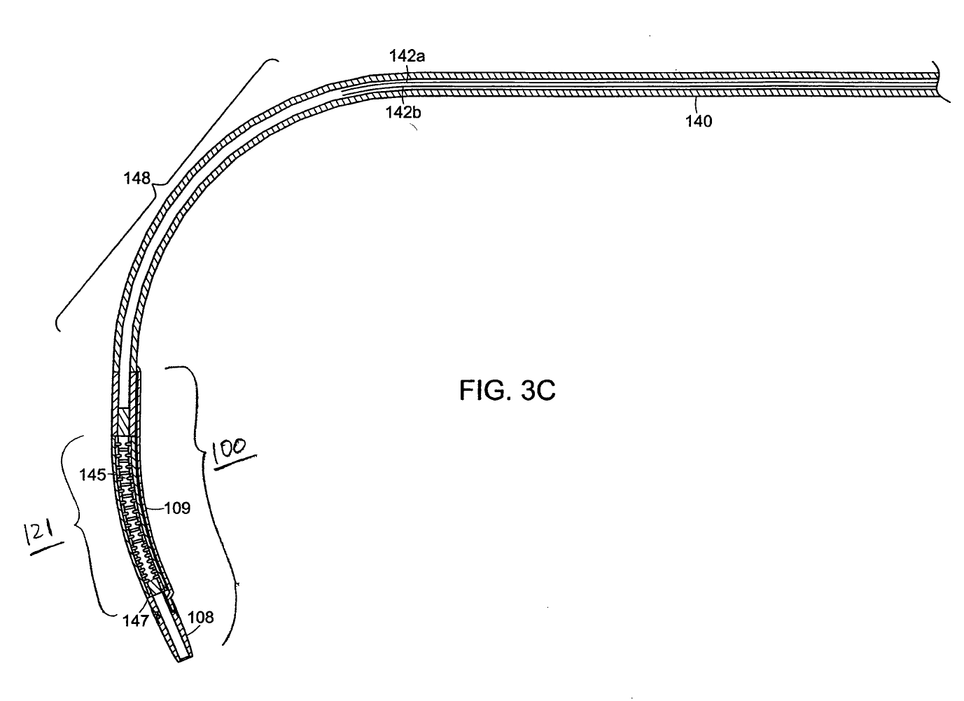

FIGS. 3A, 3B and 3C show a steerable medical instrument 136 of applicants' invention in the form of a multidirectional sphincterotome, in which the end effector is customized for a sphincter cutting operation. It will be appreciated that the following description of the sphincterotome may be readily adapted to steerable medical instruments having other types of customized end effectors, including but not limited to ERCP cannulas, stone balloon catheters, balloon dilatation catheters, and endoscopic graspers, baskets, snares, specimen retrieval devices, or a wound closure devices.

In FIGS. 3A-3C, the instrument includes an end effector 100, a shaft 140, and a control handle 144. The shaft 140 can be a flexible shaft or a rigid shaft, depending upon the application for it is designed. The shaft may be made of any material suitable for medical use, for example, polyetheretherketone (PEEK), polytetrafluoroethylene (PTFE), fluorinated ethylene propylene copolymer (FEP), urethanes, or polyether block amid (PEBA), stainless steel, polycarbonates and acrylonitrile butadiene styrene (ABS). PEBA is preferred because of its lubricity. Depending upon the application for which the instrument is designed, the shaft and end effector will have one or more lumens, shaped and sized for a given purpose. For example, in a sphincterotome, the shaft may have a guide wire lumen, a lumen for contrast medium, lumens to house the steering control wires, balance lumens, and a cutting wire lumen.

As shown in FIG. 3C, the end effector has a region 121 where the stress and strain of bending moments is distributed along the length of the region. Hinge elements may be distributed along the length of a flex beam 145, which is incorporated into the end effector. The hinge elements are optimally engineered to distribute stress over the length of the beam. Generally there are more hinge elements in the plane where there will be the greatest freedom of movement (e.g., in the cutting plane of a sphincterotome). The spacing between the hinge elements can be varied along any bending plane. In general, a hinge element may take the form of a groove, a slot, a spiral slot (screw thread form), or any other structural element that functions as a hinge.

In general, the end effector can be manufactured by injection molding. Alternatively, hinge elements can be laser cut into an end effector (e.g., into a Nitinol beam incorporated into an end effector). In yet another embodiment, hinge elements can be machined into the end effector.

FIG. 3B shows a portion of the shaft 140 and the end effector 100 of the steerable medical instrument 136 of FIG. 3A. In this embodiment, the end effector includes a molded flex tube (referenced in FIG. 3C as 145), an insulated cutting wire 109 and a distal tip 108. The end effector 100 is manufactured and engineered first as a separate component from the shaft 140. Later in the manufacturing process, the end effector and shaft are fixedly attached at 143. In a preferred embodiment, a sleeve is disposed at the lap joint between the end effector and shaft to prevent the leakage of contrast fluid and to join the end effector to the shaft. In contrast to prior art designs in which the entire instrument is formed from a single extrusion, applicants' two-component design allows for precise tip control.

Each of the steering control wires in the shaft and/or end effector may be housed in a thin-walled PTFE tubing sleeve 146 to reduce friction and to help provide precise tip control. In addition, the flex tube may be covered in whole or in part with an elastomeric sleeve 147 made of urethane, silicone, styrene-ethylene-butylene-styrene (SEBS), or thermoplastic elastomer (TPE). The sleeve functions to keep contrast media from leaking from the end effector, and has the additional advantage of generating a composite material, which helps the flex tube to resist kinking and bending forces while the elastomeric sleeve allows for flexibility.

The shaft 140 may include one or more elements (such as a wires, fibers or slugs of metal, polymer or glass) having a higher modulus than the modulus of the shaft to transmit control forces in the instrument. FIGS. 3B and 3C illustrate a preferred embodiment in which two metal stiffening wires 142A and 142B are co-extruded in the shaft 140. The co-extruded stiffening wires 142A and 142B can be made of stainless steel, carbon fiber, PEEK, polycarbonate, ABS, or glass fiber. In some embodiments, the co-extruded stiffening wires terminate prior to the pre-curve of the shaft 148 (as shown below in FIGS. 3B and 3C). In other embodiments, the co-extruded stiffening wires extend through the pre-curve. It may be desirable to extend the co-extruded stiffening wires through the pre-curve if the steering control wires are made of a monofilament or braided wires.

In some embodiments (not illustrated here), ink may be applied to or incorporated in the shaft to indicate to the operator where the cutting wire 109 exits the shaft. The ink can be a marker made out of Teflon.

FIG. 3C shows the end effector 100 connected to the distal end of the shaft 140, according to an illustrative embodiment. The shaft 140 can be a flexible shaft where the distal end can include a pre-curve 148 mechanically formed in the shaft. The function of the pre-curve 148 is to optimize the orientation of the end effector as it emerges from the exit port of the viewing scope. In a preferred embodiment, the internal wires running through the desired pre-curve section of the shaft are rolled over a mandrel to obtain a curved shape. In the embodiment of FIG. 3C, the internal wires forming the pre-curve (specifically, the cutting wire and steering control wires) are not shown. It is also possible to use the co-extruded stiffening wires in the pre-curve section. When the instrument is advanced through the working channel of an endoscope, the mechanical pre-curve must flatten out so that the instrument can be advanced through the channel. Accordingly, the wires forming the pre-curve 148 must have sufficient motion capability to allow the instrument to flex into a straight position during insertion and passage through the endoscope. When the instrument emerges from the scope's exit port, the internal wires within the instrument spring back to their original pre-curve formation. The use of a mechanically formed precurve 148 has advantages over prior art pre-curves formed by heat treating the catheter shaft.

In general, the pre-curve 148 forms an approximately 45° to 90° bend in the instrument. The bend radius of the pre-curve should be tighter than the bend radius of the channel from which the instrument is delivered (e.g., the bend radius of the working channel in an endoscope). Preferably, the bend radius of the pre-curve is less than about 1 inch.

In FIG. 4A, the flex tube 150 of the end effector has a plurality of hinge elements in the form of notches 149 made in the wall of the flex tube material. The flex tube in this illustrated embodiment is a laser cut Nitinol flex tube. Alternative flex tubes in this or any of the embodiments described herein may be made of polypropylene, polyethylene, polyetherimide (PEI), polycarbonate, polyethylene terephthalate (PET), PEEK, or a nylon material. A steering control wire 152 extends through the flex tube 150. More than one steering wire may be present in the end effector depending upon how many planes of motion are desired in the instrument. Bands of heat-shrink material 156 (such as PET) are disposed at predetermined locations along the flex tube 150. In addition to the traditional role of providing distance markers along the cutting wire 109, the heat-shrink bands may be employed to maintain and hold the position of the steering control wires within the end effector. The flex tube 150 can be encased by a sleeve 160 to seal in contrast fluid. In a preferred embodiment, the flex tube 150 is made of polypropylene and the flex sleeve 160 is made of urethane.

FIGS. 3A-3C and FIG. 4A illustrate embodiments of Applicants' steerable instrument that comprise a cutting wire 109. In addition to the steering control wires 152, the cutting wire 109 may be used for steering the instrument in the cutting plane. In these embodiments, the cutting wire 109 extends from the user handle 144 distally through the shaft 140 to a point near or within the end effector 100, at which point the cutting wire exits the inside of the shaft 140 through a sidewall port and runs distally along the outside of end effector to a point proximal to the tip 108 of the end effector 100, at which point the bowing wire 109 enters another sidewall port (as illustrated in FIG. 4B) and is anchored inside the end effector 100. In some embodiments, the cutting wire exits at the proximal end of the end effector. Tension applied to the cutting wire 109 pulls the cutting wire taut and causes the distal end 108 of the end effector 100 to flex in the direction of the applied tension. In Applicants' invention, the end effector can flex from 0° to about 180° in the primary plane of bending (e.g., the cutting plane), and preferably from about 80° to about 110°. In other embodiments, the instrument may have a left steering control wire and a right steering control wire in addition to the main bowing or cutting wire. Tension applied to the left steering control wire causes the distal end 108 of the end effector to bend left. Tension applied to the right steering control wire causes the distal end 108 of the end effector 100 to bend right. Left and right steering control wires, in Applicants' invention, may create up to about ±90° of motion from the primary plane of bending, and preferably from about ±25° to about ±45°. In alternative embodiments, the steerable instrument may have four steering control wires (up, down, left and right), but no cutting wire.

In some embodiments, the tip 108 of the end effector is a rigid tip attached at the distal end of the flex tube 150. The tip 108 can have a smooth, rounded geometry that facilitates atraumatic cannulation. In some embodiments, two lumens can exit the tip 108, namely a guide wire lumen and a second lumen for contrast media injection. Alternatively, the tip 108 can contain a single common exit port for both the guide wire and the contrast media. The tip 108 can be manufactured using any recognized technique suitable for medical devise manufacturing, including injection molding.

As in FIG. 4A, the flex tube 150 of the end effector is located in a region proximal to the tip 108 of the end effector. An eccentric load can be applied to the one or more control wires 152 to articulate the end effector out of the plane of the cutting wire 109. The material characteristics of the flex tube 150 can prevent the end effector 100 from buckling or kinking under this stress. The flex tube 150 can be coated with an electrical insulating material, such as Parylene C, a di-para-xylene-based polymer coating provided by Parylene Coating Services, Inc. of Katy, Tex., Teflon, TetraFluorEthylene-Perfluorpropylene (FEP), Polytetrafluoroethylene (PTFE), or Polyimide. In some embodiments, the insulated flex tube is further encased in a sleeve of flexible material 160 to provide a smooth exterior surface. The sleeve 160 may be formed from silicones, urethanes, styrene-based copolymers such as styrene-ethylene-styrene block copolymer (SES), and thermoplastic elastomers such as Kraton™, Pebax™, and Sanoprene™.

In some embodiments, a flex tube may be formed by making a spiral cut into the distal end of the shaft of the biliary catheter.

FIG. 4B shows one embodiment for attaching the flex tube 150 to the tip 108 of the end effector 100. The flex tube 150 can be recessed concentrically inside the tip 108, with the flexible sleeve 160 abutting the tip 108. Adhesives or heat shrink can be applied at the lap joint 164. In some embodiments, the surface of the shaft 140 and the end effector 100 are treated prior to adhering or attaching the shaft to the end effector 100. The surface can be treated using etching, plasma or corrona. The proximal end of the flex section can be joined to the distal end of the shaft 140 in a similar fashion.

FIG. 4C shows a steering control wire 152 running through the flex tube 150 according to an illustrative embodiment. In the case of a sphincterome, with the cutting wire 109 located in the 12 o'clock position, the steering control wires 152 can be located anywhere between the 12 o'clock to 6 o'clock range, and the 6 o'clock to 12 o'clock range, respectively. In some embodiments, the control wires 152 are placed at about 110-120° radially on either side of the bowing wire 109. Adhesive can be used to bond the cutting 109 and steering control wires 152 into the tip 108 of the end effector 100. In some embodiments without a cutting wire, there are four steering control wires at 3 o'clock position, 6 o'clock position, 9 o'clock position, and 12 o'clock position.

FIG. 4D shows the different elements of an end effector 100. A plurality of heat shrink bands 156 can be employed around the flex tube 150 to create “eyelets,” which can maintain the control wires 152 in proper alignment in the flex tube 150. Alternatively, a single piece of heat shrink can be spiral cut and employed around the flex tube 150 to secure the control wires 152 in proper alignment. In some embodiments, the tip 108 is provided with a hydrophilic coating to ease cannulation. A sleeve 160 can be used to seal contrast fluid in the end effector. The sleeve 160 can be made of urethane.

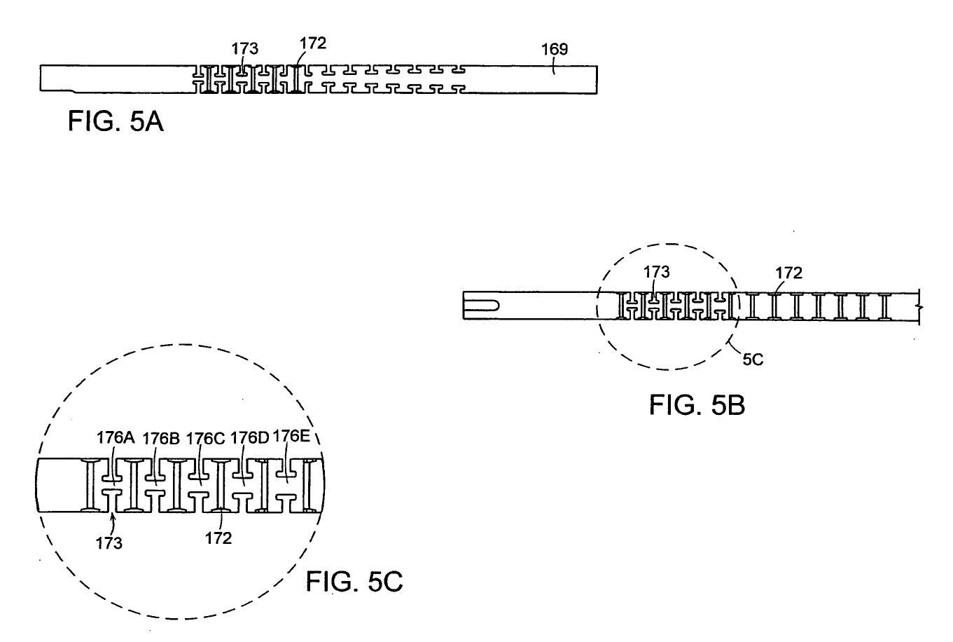

FIG. 5A shows the hinge elements in a flex tube 169 of an end effector according to another illustrative embodiment. The hinge elements in the flex tube 169 can be disposed at different points along the longitudinal axis of the end effector to change a material property such as stiffness. In this embodiment, the hinge elements are “T-bar” notches 173. T-Bar shaped notches 173 can reduce stress concentrations and improve flexing fatigue. In some embodiments, the flex tube 169 is injected molded to generate a varying pattern of notches to change the stiffness of the flex tube 169 along its longitudinal axis. In some embodiments, the end effector has a region where the stress and strain of bending moments is distributed along the length of the region. In this embodiment, the spaces between the T-bar notches are larger at the proximal end of the end effector and taper down to become smaller and smaller as one nears the distal tip. The orientation, disposal and spacing of the hinge elements can be varied along any bending plane.

In some embodiments, the end effector can experience a greater bending moment at the proximal end of the end effector and a lesser bending moment at the distal end during use. Generally there are more hinge elements in the plane where there will be the greatest freedom of movement (e.g., in the cutting plane of a sphincterotome). The distal end of the flex tube 169 can have a greater density of hinge elements than the proximal end of the flex tube 169 to account for the variable bending moments. In some embodiments, changing the density of hinge elements in the flex tube 169 allows greater flexibility at the distal end of the flex tube 169 and the end effector while accounting for the increased bending moments experienced at the proximal end. By increasing the spacing between the hinge elements at the proximal end of the flex tube 169, the proximal end of the flex tube 169 can withstand the greater bending moments. The design also has the effect of distributing stress over the length of the beam instead of concentrating the stress and strain which can lead to kinking and compromise the structural integrity of the end effector. In some embodiments, notches can be placed perpendicular to one another. For example, in FIG. 5A, notches 172 are disposed perpendicular to T-bar notches 173. This configuration can account for bending moments and distribute strains and stresses experienced in the end effector in multiple planes. A plurality of notches can be disposed at different angles relative to one another to account for bending moments in multiple planes. The notches can generate a more even distribution of stresses and strains in multiple planes.

FIG. 5B shows an alternative pattern of hinge elements, wherein T-Bar notches 173 are used in the distal end of a flex tube but are not used in the proximal end.

FIG. 5C shows yet another pattern of hinge elements that can be used in a flex tube. In this embodiment, the spacing between the notches 176A to 176E perpendicular to the longitudinal axis of the flex tube are varied to account for the changing bending moments in the flex tube 169. In this embodiment, the spacing decreases as one moves in the distal direction. Varying the spacing between the T-Bar shaped notches can vary the stiffness of the molded flextube along its longitudinal axis. T-Bar shaped notches can be useful if the flextube is made of Nitinol, to account for the brittle nature.

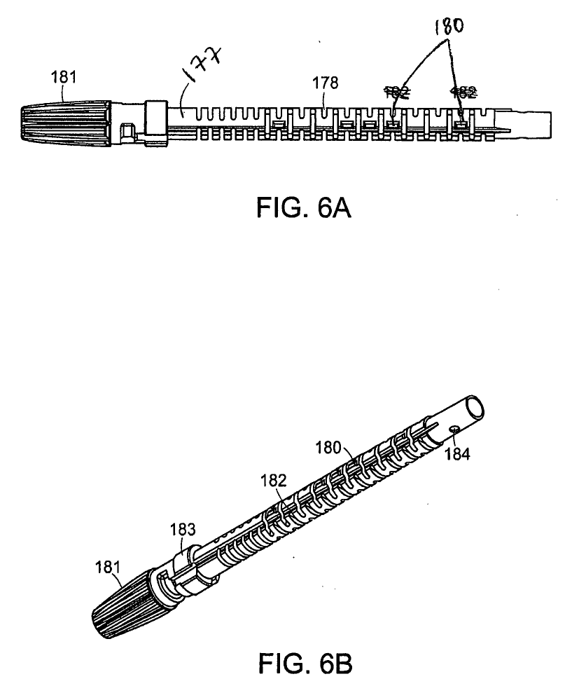

FIG. 6A shows a molded flextube 177 according to another illustrative embodiment. In some embodiments, the end effector is not a flex tube attached to a separate tip 108, but is instead an integrally molded flextube 177. The molded flextube 177 can be injection molded to have notches 178 variably disposed along its length to account for bending moments. Simple notch geometry, as shown here, may be acceptable for semi-rigid plastic.

In the illustrated some embodiments of FIGS. 6A and 6B, the distal end of the molded flextube forms a fluted tip 181. The fluted tip can reduce the frontal area of the tip and reduce trauma. In some embodiments, the fluted tip 181 reduces the force required to enter the sphincter. The fluted tip 181 can be molded in one piece with the flex tube, or may be separately made and attached.

As illustrated in FIG. 6B, the molded flextube may include a steering control wire channel 182 in the flextube to provide support and maintain control wire positioning during flexing. The molded flextube can also have a concentric element 183 that protrudes out and acts as an interface with another device. Guides 180 are provided in order to prevent displacement of the steering control wires. The hole 184 in the proximal end of the end effector can mate with a hole drilled in the distal end of the shaft, for joining the two parts. A mandrel may then be inserted through the assembly and glue can be injected to join the end effector, the mandrel preventing glue from encasing the lumen.

FIG. 11A shows an alternative end effector comprising a flexible spring region 344 and a distal end cap 348. A cutting wire and two steering control wires (352 and 353, respectively) run through the shaft 140, exit the shaft and run outside the end effector over the spring region, reenter the instrument at a point near the distal tip, and are anchored inside at or near the distal tip. To reinforce the device under cutting wire tensioning loads, the end effector utilizes a simple compression spring 356 and an end cap 348. The end cap 348 can be made of metal or plastic. The left and right tensioning wires can be covered with a material that can prevent tearing or abrasions during insertion of the device and bowing of the bowing wire. A silicone covering material may be bonded onto the endcap 348. Judiciously spaced wire guides may be disposed along the axis of the spring 356 to prevent the steering control wires from passing the center of the compression spring 356 during loading. The lumen 360 is stiffened with a metallic based oversheath to reduce lumen compression during loading. The compression spring 356 may comprise one continuous spring or spring segments. Intermediate wire guide spacers can be used along the length of a continuous spring or at the ends of spring segments that can allow the range of flexibility for the device to reach beyond the −45°-0°-+45° range.

FIG. 11B illustrates yet another embodiment of an end effector, here applied to a sphincterotome. The instrument comprises two control steering wires (left and right, respectively) 364 and a third wire, which is the bowing or cutting wire 368. The three wires can extend from a point of attachment in the control handle of the device to a point near or at the distal end of the device. Preferably, the three wires can be spaced around the longitudinal axis of the catheter at 120° intervals. The device can also include a concentric guide wire lumen 372 located on the longitudinal axis of the catheter. The guide wire lumen can also be used to deliver contrast media. Alternatively, the surgical instrument can include a separate contrast media lumen. The left and right steering control wires may be anchored in the distal end of the device by wrapping the wires around the outer wall of the guide wire lumen as shown at 376. The distal end of the cutting wire 368 is formed into an integral compression spring. In this design, the distal end of the integral spring component has an end moment load exerted on it which cases a curvature. The deflection capabilities of the integral spring component enable the distal tip of the end effector to handle very tight curvatures without permanently deforming the material. The compression spring structure allows multi-directional displacement and compressive rigidity. Because the bending stiffness in the spring configuration is lower than the bend stiffness in conventional sphincterotomes, wherein the cutting wire is anchored at the distal tip by way of a rigid connection, the integral spring effector reduces the forces required to actuate the tip. Furthermore, the integral bowing wire 368 and spring tip construction eliminates a number of components and assembly steps such as welding, crimping and bonding in a small area. The coil spring 380 can provide the catheter with a high degree of kink resistance. The integral spring can keep the catheter body in circular form, even if localized intercoil wall kinking might occur, so that the guide wire can pass through the guide wire lumen 372 in a tight bend. The tensioning wire wrapped anchor 376 has a lower profile and is less costly to manufacture than existing T-tube wire anchor techniques. Conventional devices utilize a T-anchor that is crimped, welded and inserted into the distal end of the bowing wire lumen. This design is costly, space-consuming, and tedious to assemble.

As shown in FIG. 11C, the spring and left-right tension wires 364 can be covered at the distal end of the device by an electrical insulating material 384, such as a silicone elastomer. The insulating material 384 can protect the adjacent tissue and to keep the tension wires 364 near the spring wires, particularly in severe bend conditions when the wires may pass the centerline of the spring. The elastomeric insulating material 384 can function to keep the wires in the correct position, and can deflect enough so that sliding motion between the wires and elastomer surface is not required.

In some embodiments, tip of the device is a hard PTFE tip, for example by using a conventional catheter tipping process. The spring can be covered with an elastomer or other insulating material 384 for electrical insulation. In another alternative embodiment, a convoluted PTFE shrink tube is placed over the spring coils for friction resistance and electrical insulation. In some embodiments, silicon elastomer is placed over the spring coils. The convolutions allow the shrink tube to flex with only a moderate effect on the actuation loads and tip rigidity. Integral molded silicone insulation 384 can be less traumatic than a hard PTFE shaft tip cut at a right angle.

The use of insulation elastomer 384 at the distal tip can provide for a high voltage yet low mechanical stiffness insulation method. Using a small diameter PTFE section along with a linear spring can prevent the shaft from kinking and provides a catheter with more repeatable arcing motion from the tip.

In some embodiments, the left-right tensioning wires 364 are overmolded adjacent to the integral flex spring 380. This construction allows the wires to move moderately relative to the coil springs. During the tensioning action, the wires tend to move away from the coil spring, increasing the moment arm and effectively decreasing the applied load for a given angular deflection and preventing “neutral axis wire crossover.” In some embodiments, the wires do not cross the neutral flex axis of the system, allowing the opposite tensioning wire to bring a deflected tip back to a neutral position.

A device having a spring tip 384 end effector may be manufactured by, for example, cutting the catheter shaft to length, reducing the tip diameter using conventional heated, drawn-down dies and core pins, and trimming back the length of the reduced tip. The bowing wire 368 and guide wire lumens 372 can be skived. The injection lumen can be skived over to the guide wire lumen 372. In some embodiments, the bowing wire 386 is fed into its respective lumen. The bowing wire 368 tip can be formed into a compression spring 380 using a bench top spring winder. The spring can be assembled over the reduced diameter tip. The left-right tensioning wire 364 can be cut to length and the loop ends are fed into the tensioning wire lumens in the catheter shaft. The end of the left-right pull wire can be formed into a retaining loop 376 using a device similar to a bench top spring winder. The formed left-right wire anchor can be assembled onto the distal end. The tip can be overmolded with, for example, an insulation elastomer 384 such as silicone elastomer, and flexed. The distal tip 388 can also be formed from, for example, PTFE from the catheter extrusion itself, to provide a hard, atraumatic surface for cannulation.

FIG. 12A shows a flex beam end effector 392, according to an illustrative embodiment wherein the end effector comprises a Nitinol flex beam 396 and an elastomer overmolded flex section 400. A Nitinol flex beam 396 can utilize its super-elastic portion of the stress strain curve to allow flexure and still return to a nominal center position. In some embodiments, the flex beam end effector 392 has control wires 404 and a bowing or cutting wire 408.

A flex beam 396 made of Nitinol can achieve an extremely tight radius of curvature without failure. Nitinol material in the appropriate heat treatment and alloy can exceed the elastic strain limitations of ordinary steels and metallic materials, allowing for greater deflection than would normally be possible. The actuation forces are substantially lower with a Nitinol flex beam operating in the super-elastic region. Lower actuation forces tend to decrease control system losses and allow for a more sensitive control feel. The flex beam 396 can achieve an extremely tight radius without failure. In the super-elastic region, the stress strain curve of Nitinol is essentially flat from 1% strain to 8% strain, which can translate into high tip deflections with no additional motion resistance.

In some embodiments, the flex beam end effector 392 has a continuous guide wire lumen 398, which provides for a burr-free guide wire path. This can reduce the drag of the guide wire from burrs and sharp edges, thereby facilitating the cannulation process. During the cannulation process, the user can “feel” when the guide wire touches tissue. Additional resistance or burrs can cause a “mis-read” of the guide wire/tissue contact.

The small, frontal cross-section and overall low profile of the flex beam end effector 392 reduces the required cannulation forces, particularly if a user attempts to cannulate a “tight” papilla. In some embodiments, the surgical instrument is a biliary catheter, including a flex beam 396 that provides an improved cannulation process that minimizes the number of unsuccessful cannulation attempts, which are well-known for causing pancreatitis.

Manufacture of the flex beam end effector 396 can require little wire forming. In some embodiments, a catheter extrusion 397 is cut to length, a counter-bore is made on the guide wire axis. A center guide wire lumen can be cut and skived to allow contrast passage at a cross-over hole 424.

As shown in FIG. 12B, the bowing wire 408 can be attached to the flex beam 396 using a cylindrical crimp tube 395. The tip of the end effector 412 can be overmolded with a polymer. A Nitinol flexbeam wire can be crimped to the bowing wire and steering control wires 404 at the distal end of the flex beam end effector 392. The tip 412 can be overmolded with a hard polymer to capture the wires and flexible tube 396 in their correct orientation. The stationary end of the flex beam 396 and tube 397 can be mounted into a shaft 428 using either a force fit and/or by bonding. The tube 397 can be used to unite the overmolded components. The steering control wires 404, flex beam 396, and tube 397 can be overmolded with a silicone elastomer 413 for insulation. The control wires 404 can be threaded through the shaft extrusion, and adhesive is applied to connect the tip assembly. A slit can be cut in the extrusion and the bowing wire 408 is fed through the opening of the catheter to complete the tip assembly.

FIGS. 13A and 13B together illustrate an integral spinal tip end effector 444. The integral spinal tip 436 can include a plurality of hinge elements in the form of spinal flextures 440, which allow bending along their “weak” axes, yet are stiff in compression. This configuration allows moment loads to be easily generated with small pull wire motions. The spinal tip 436 can be a single core component, which can be manufactured, for example by injection molding. In some embodiments, the wire anchoring method is simplified with no welding, bonding or critical processes. The overmolded, atraumatic tip may be added, and can achieve support from the underlying integral spinal structure. In this embodiment, as well as the other various embodiments described herein, separate guide wire and contrast media passages can merge proximal to the tip of the end effector. This allows the guide wire to stay substantially free of contrast media. When certain contrast solutions, for example those that are barium-based, flow along the length of the guide wire lumen, the solution can cause the guide wire to have a ‘gritty feel’, thereby de-sensitizing the cannulation process for the user. In some embodiments, the merger of the guide wire lumen 460 and contrast passages is accomplished on the proximal side of the flexible section 456 of the catheter so that the tip size can be reduced. In some embodiments, the passages merge into a single lumen at the distal end of the shaft. Cannulation can be easier to accomplish with a smaller sized catheter tip.

The integral spinal tip end effector can be manufactured through a series of steps. The catheter shaft 456 can be cut to length. A spinal tip 436 can be inserted into the guide wire lumen 460. The material for the molded spinal tip 436 can be, for example, a high temperature material such as FEP. The tip assembly can be overmolded. In some embodiments, PTFE is preferred around the bowing anchor and extrusion exit skive locations. The bowing wire 464 and left-right pull wire loop 468 can be inserted into their respective lumens. As shown in FIG. 13B, the distal end of the bowing wire 464 can be wrapped around the tip and looped around itself. The left-right pull wire 468 can be wrapped around the end of the tip.

As shown in FIG. 13C, the flexible portion 436 of the tip of the end effector 444 may be overmolded with an elastomeric material 472. The tip of the end effector 444 can be overmolded using silicone, SEBS, urethane, or other materials suitable for the flex sleeve of the end effector. The inside of the tip can be relieved to allow passage of a guide wire. The integral spinal tip 436 allows for flexibility in two planes and stiffness in compression, which results in a more sensitive device because the tip is not deflected axially (higher axial spring rate). This can be an advantage during cannulation where manual dexterity and “feel” is important.

The injection lumen overmolding core can be inserted into the injection lumen. The guide wire lumen core can be inserted into the end effector. The end effector can be overmolded 472 and the cores can be removed and tip flexed. In some embodiments, the pull wires 468 are some distance from the underlying structure and can remain attached to the elastomer 472, yet deflect the far field elastomer 472 to achieve their function. This can simplify the overmolding process and eliminates a number of complex coring operations.

FIG. 14 shows a segmented end effector 480 according to an illustrative embodiment. A segmented end effector 480 can include segments 484 A-J in the distal section. In some embodiments, the segments are about 0.1 of an inch long. The segments can be molded from plastic with discrete lumens for the guide wire, tensioning wires, bowing or cutting wire and optionally contrast media. In some embodiments, the guide wire lumen is lined with a polyimide material that improves the alignment of the segments 481 and provides a channel for distal dye injection. This embodiment provides for a flexible distal section. The segmented portions of the end effector 480 can be molded, machined or extruded by known methods.

In yet another alternative embodiment, the end effector comprises a tapered beam, where the diameter of the beam is larger at its proximal end and tapers to a smaller diameter as one approaches the distal end of the beam. The beam is designed to be thicker at the proximal end because this is where the beam experiences a higher bending moment. This design is feasible for simple embodiments of applicants' medical instrument that do not require many lumens and wires to be located in the end effector.

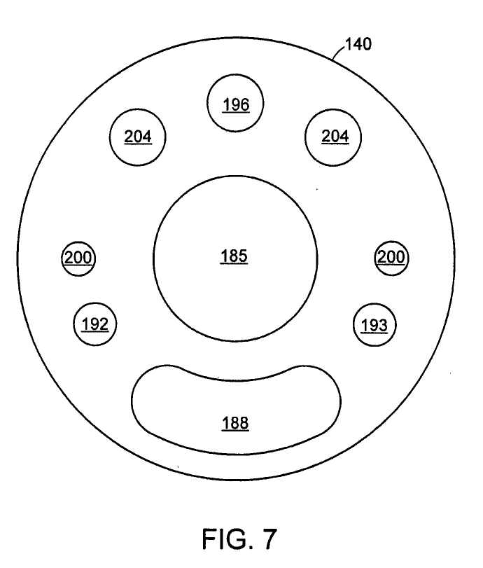

FIG. 7 shows a plurality of lumens in a shaft 140, according to an illustrative embodiment. The multi-lumen shaft can be made out of Teflon™. In this embodiment, the shaft has a guide wire lumen 185, a dedicated contrast media lumen 188, first and second steering wire lumens 192 and 193, and a cutting wire or third steering control wire lumen 196. In a preferred embodiment, the bowing wire lumen 196 is in the 12 o'clock position, the steering wire lumens 192 and 193 are positioned at approximately the 4 and 8 o'clock positions, the contrast lumen 188 is opposite lumen 196, and the guide wire lumen 185 is in the center. As illustrated in FIG. 7, the shaft may further include two stiffening wire lumens 200 at the 3 and 9 o'clock positions. The stiffening wires aid the shaft 140 in transmitting motion from the control handle of the device to the tip 108 of the end effector 100 and can allow the user to maintain precise control. Orienting the stiffening wires in the 3 and 9 o'clock positions predisposes the device to bend in the 6 and 12 o'clock positions, which enables the operator to better control the tip orientation as the device exits the scope. The ability to control end effector orientation out of the scope can facilitate easier cannulation. Stiffening wires also can prevent spiraling of the shaft during extrusion. Finally, as illustrated in FIG. 7, the shaft 140 may include balancing lumens 204. The balancing lumens 204 can be used to achieve pressure stabilization of the shaft 140 generated by the other lumens.

In some embodiments of applicants' steerable medical instrument, it will be desireable to merge the separate guide wire and contrast media lumens into a single lumen at a point proximal to the distal end of the instrument. To accomplish this, the internal wall between the guide wire lumen 185 and the contrast lumen 188 can be cut away so that the two lumens merge and contrast media enters the guide wire lumen 185 and exits at the tip of the end effector. The merged guide wire/contrast lumen can run over a distal 20-25 mm of the instrument, minimizing the disruptions on the surface of the tip of the end effector. This configuration allows for a single edge created on the central axis of the device by the merged guide wire/contrast lumen exiting the distal most end of the tip of the end effector. In some embodiments, the configuration of merging the guide wire/contrast lumen enhances hydrostatic device exchanges.

In some embodiments, a stylet is used in the guide wire lumen 185 of the shaft 140 to fill in the distal exit port, to generate a smooth, continuous, edge-free surface at the tip to ease cannulation. In one embodiment, a polymer stylet is employed. In another embodiment, a pre-loaded guide wire is indexed like the stylet for initial cannulation. In yet another embodiment, a needle knife stylet is employed.

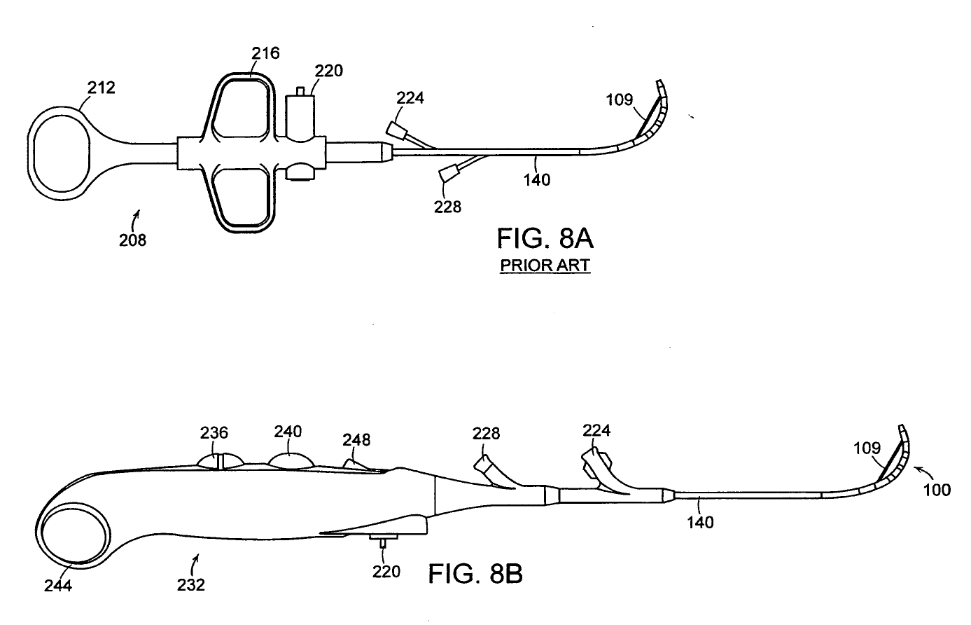

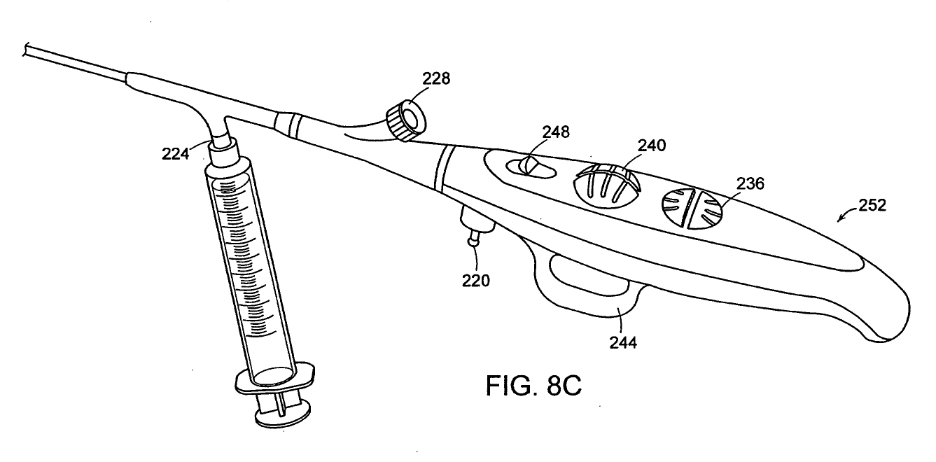

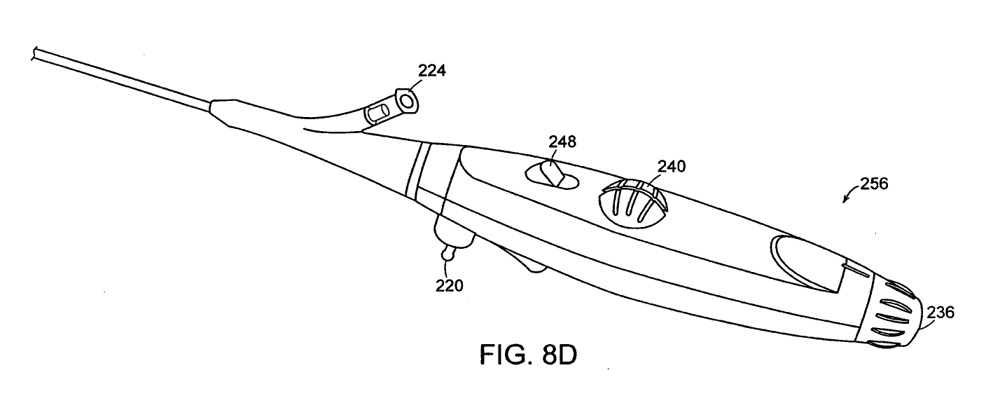

FIG. 8A shows a prior art control handle 208 with a rotatable thumb loop 212 to steer the surgical instrument and a bowing control element 216 to control a bowing or cutting wire 109. The control handle 208 also includes an electrode connector 220, a contrast port 224 to inject contrast media, and a guide wire port 228. There is a shaft 140 connected to the control handle 208 and the shaft is shown in a bowed configuration.

FIG. 8B shows a control handle assembly according to an illustrative embodiment of Applicants' invention. The control handle 232 has an anatomically shaped handle including a multi-directional control 236 in the form of a rounded wheel. Rotating the multi-directional control 236 to the operator's right moves the tip 108 of the end effector 100 of the device to the right. Rotating the multi-directional control 236 to the operator's left moves the tip 108 of the end effector 100 to the left. As illustrated in FIG. 8B, the control handle 232 assembly can also include a bowing/cutting control 240 that can be in the form of a rounded wheel. Rotating the bowing/cutting wheel 240 in the proximal direction tensions the bowing/cutting wire 109 and causes the device to bow in the cutting plane. In some embodiments, the multi-directional control 236 and the bowing/cutting wheel 240 are coated to increase traction during use.

The control handle assembly can also include a finger ring 244 at the proximal end of the handle assembly. The finger ring can provide an anchor or point for grounding the device in the operator's hand.

The control handle 232 can also include a braking control device 248. In one embodiment, the braking control 248 is in the form of a push-down button, which may be turned on or off as the operator desires. If the operator activates the braking control 248 by pushing down on the button, the operator can then actuate the bowing control wire 109 and the end effector 100 will stay in the position where it is placed. Alternatively, the braking control feature can be a constant control that is always turned on. In some embodiments, the handle can have a friction control pad to activate and deactivate braking control.

FIG. 8C shows an alternative embodiment of a control handle 252. As in this embodiment, the finger ring 244 can be placed under the control handle and the contrast port 224 can be located under the control handle.

FIG. 8D shows an alternative control handle 256 where the multi-directional control 236 device is located at the proximal end of the control handle. In some embodiments, the multi-directional control device is a joystick that can be manipulated by the user to split the wire tension of the control wires. The handle assembly can include a sliding part, which may be manipulated to apply tension to the bowing wire 109. In some embodiments, an elevator of the endoscope is used to give the unit approximately 110° of elevation.

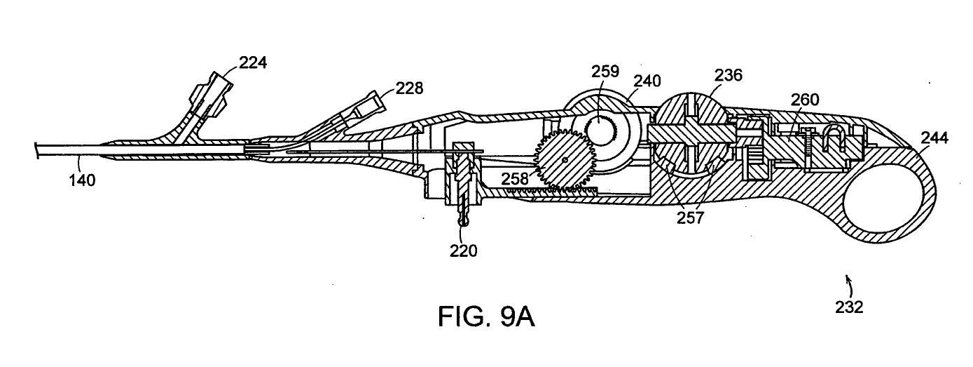

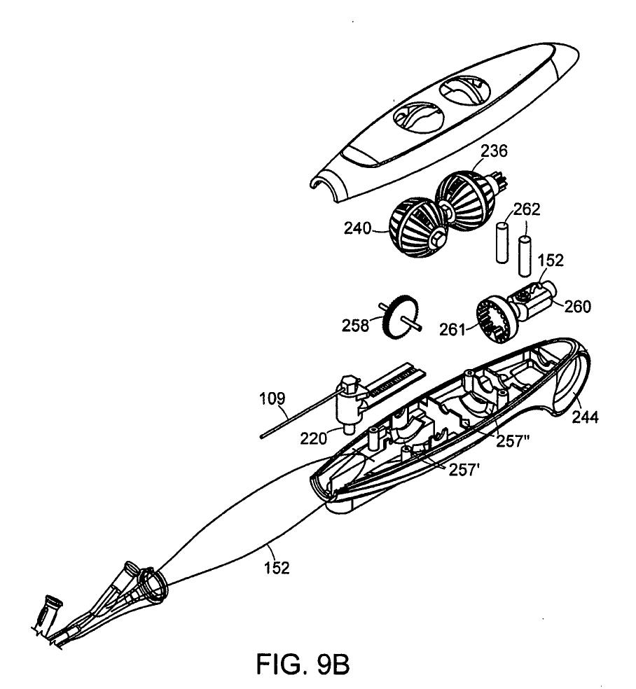

FIG. 9A shows how foam pads 257 can interact with a control surface of the multi-directional control device 236 for “braking” of the instrument. The foam pads 257 can exert a frictional force on the multi-directional control device 236. In some embodiments, the frictional force exerted by the foam pads 257 “brakes” or restrains movement of the device unless a rotational force is exerted by the user on the multi-directional control device 236. In some embodiments, the foam pads 257 are made of a silicon-based or urethane-based foam. The foam pad can be an open cell foam or a closed cell foam. In some embodiments, the foam is made of a low compression set foam that does not take a set over time.

In some embodiments, a series of gears 258 and 259 can be used to control a bowing wire. The gear teeth can be removed 259 to provide a “neutral position” for bowing wire control. This can allow the device to be coiled for packaging without overstressing the tip. The gear profile “filled” in provides precise bowing limits. This can prevent users from breaking or kinking the device by over-actuating the bowing wire 109.

FIG. 9B shows how foam pads 257′ and 257″ can interact with the multi-directional control device 236 and a bowing/cutting control device 240, according to another illustrative embodiment. The foam pads 257′ and 257″ can provide braking action on the handle controls. This can allow the user to leave the device in a preferred position even when moving from one control to another. Unless the user exerts a force on the multi-directional control device 236 or the bowing control 240, the device remains in the position. In some embodiments the control wire 152 can be a control wire loop instead of separate control wires.

As shown in FIG. 9B, the steering control wires 152 can be actuated using a “drum ring gear” 260. In some embodiments, when a user exerts a force on the multi-directional control 236, it actuates a gear 261 that actuates a drum ring gear 260 which manipulates the steering control wires 152. The drum ring gear 260 can rotate 180° and works as a tension control system. When the drum rotates in a first direction, one steering control wire tenses and the other steering control wire relaxes. In some embodiments, roller pins 262 are placed to guide the control steering wires from the drum ring gear 260. In some embodiments, stationary pins are used.

FIG. 10A demonstrates how the steering control wires 152 can be attached to the control handle. In this embodiment, the multi-directional control 236 actuates the control wire by the use of a bevel gear with a pulley. A user can rotate the multi-directional control 236 in a first direction that is connected to a first gear 263 that engages with a second gear 264. The second gear can include a pulley 268 that manipulates the control wire 152.

FIG. 10B shows the use of a double helix configuration to manipulate a left control wire 152′ and a right control wire 152″. In this illustration, the multi-directional control 236 is connected to a helical camshaft 272. The helical camshaft 272 can be connected to two carriers 276 that actuate control wires 152′ and 152″. The user can rotate the multi-directional control 236, which can rotate the helical camshaft 272. The carriers 276 can follow the tracks 280 in the helical camshaft 272, manipulating the control wires 152′ and 152″.

FIG. 10C shows a double lead screw used to actuate the control wires 152′ and 152″, according to an illustrative embodiment. The multi-directional control 236 is connected to a spur gear 284 that can be connected to two lead screws 288. Carriers 292 that actuate the control wires 152 can be attached to the lead screws 288. The user can rotate the multi-directional control 236, which can rotate the spur gear 284, causing the lead screws 288 to rotate. The carriers 292 can move along a longitudinal axis of the lead screws 288 manipulating control wires 152. In some embodiments, mechanical stops are placed on the shaft and the mechanism does not require a break.

FIG. 10D shows a beaded chain mechanism, according to an illustrative embodiment. The multi-directional control 236 is attached to a sprocket driver that engages a sprocket 300. When a user rotates the multi-directional control 236, it can engage the sprocket 300 that actuates a beaded chain 304 and control wires 152′ and 152″ that attach to the beaded chain 304. Guide rails 308 can be used to control the beaded chain 304.

FIG. 10E shows a bevel gear utilized to manipulate control wires 152′ and 152″, according to an illustrative embodiment. Rotating the multi-directional control 236 can engage a miter gear 312 and a plurality of spur gears 316 and 317. Movement of a spur gear 316 can cause movement to racks 320 that can be attached to control wires 152′ and 152″. In some embodiments, mechanical stops can be placed in the rack 320 track.

FIGS. 10F-10H show a “half-spur” gear utilized to manipulate control wires 152′ and 152″, according to an illustrative embodiment wherein some of the teeth of the gears were removed. In The control mechanism allows for articulation in a first and second direction and also has a neutral/rest position. As shown in FIG. 10F, when the half spur gear 316′ is rotated in a first direction, the first control wire 152′ can be manipulated in a first direction while the second control wire 152″ is free to move. As shown in FIG. 10G, the configuration of the half spur gear 316′ allows for a neutral position. FIG. 10H shows the half spur gear 316′ rotated in a second direction, allowing the second control wire 152″ to be manipulated in a second direction while the first control wire 152′ is free to move.

FIG. 10I shows the use of a face cam mechanism to manipulate control wires 152′ and 152″. In this illustration, the multi-directional control 236 is connected to a face cam 324 with followers 328 and 328′ and follower springs 332 and 332′. In the initial position (neutral) the followers 328 and 328′ can be aligned above the centerline of the cam 324. This dimension can be defined by the required forward linear travel of the control wire 152 to allow tip motion and cam surface angle. When the cam surface drives one follower 328 back, the other 328′ can follow forward by a controlled amount and enter the dwell surface 336 on the face cam. The system can prevent slack build up resulting from extension of the control wires 152′ and 152″.

FIG. 15 shows the steps for manufacturing a steerable surgical instrument, according to an illustrative embodiment. An internal skive 488 can be performed at a distal end of a multi-lumen shaft, such as a catheter. The shaft can be marked 492 indicating where the cutting wire should exit. The proximal and distal surface of the shaft can be prepped 496 to attach to a control handle and end effector, respectively. In some embodiments, the surface is treated using a plasma, corona, or etching procedure. A counterbore 500 can be used to create a guide wire port and contrast port 504. Insulated control steering wires and bowing wires 508 can be integrated with the shaft 512.

The flextube of the end effector can be integrated with the flex section 516 which can be integrated with the tip sleeve. The tip of the end effector can be coated 520. The end effector and flexible tube can then be integrated by the use of adhesives. A pre-curve then can be mechanically formed in the shaft 524, by using the internal wires, such as the bowing wire and steering control wires. In some embodiments, the pre-curve includes at least two wires.

The parts of the control handle include the idler gear, a bowing wire knob, rack and multi-directional control 528 which can be assembled into a control handle 532. In some embodiments, the controls of the control handle are coated to increase traction to help engage the device with the user's hand. The controls of the control handle can be coated with a urethane. In some embodiments, the controls are made of a semi-rigid TPE that is not coated. The control handle can be integrated to the shaft which has been integrated with the end effector. In some embodiments, the device is sterilized prior to use 536.

A steerable medical instrument, as described above, can be positioned in a patient's body with the use of a viewing scope having a distal exit port. The scope can be navigated through the patient's anatomy and positioned near or adjacent the desired area in the patient's body. The steerable medical instrument can be introduced through the scope and advanced until the distal end of the instrument protrudes from the distal exit port of the scope. The distal end of the instrument can be steered by tensioning at least one steering control wire.

A steerable medical instrument can also be used to cannulate the Papilla of Vater in a patient. A flexible endoscope can be used with a steerable medical instrument as described above. The endoscope can be navigated through the patient's anatomy and be positioned so that the distal exit port is near or adjacent the Papilla of Vater. The steerable medical instrument can be introduced through the endoscope and advanced until the distal end of the instrument protrudes from the exit port of the endoscope. The instrument is further advanced and steered to enter and cannulate the Papilla, wherein the steering is achieved by tensioning at least one steering control wire.

While the invention has been particularly shown and described with reference to specific illustrative embodiments, it should be understood that various changes in form and detail may be made without departing from the spirit and scope of the invention.

What is claimed is:

1. A steerable medical instrument comprising:
a shaft comprising a proximal end and a distal end;
an end effector at the distal end of the shaft, said end effector comprising a proximal end and a distal end;
one or more steering control wires anchored in the end effector such that tension applied to the wire proximal to the anchor point causes deflection of the end effector in the direction that tension is applied; and
a control handle connected to the proximal end of the shaft;
wherein a material property of the end effector varies along its length to account for variable bending moments experience by the end effector when tension is applied to the one or more steering control wires.

2. The steerable medical instrument of claim 1, wherein the shaft is flexible and is configured to be delivered through a channel in a viewing scope.

3. The steerable medical instrument of claim 2, wherein the viewing scope is selected from the group consisting of an endoscope, colonoscope, bronchoscope, and laparoscope.

4. The steerable medical instrument of claim 1, wherein the material property of the end effector is stiffness.

5. The steerable medical instrument of claim 1, wherein the end effector comprises a flex tube.

6. The steerable medical instrument of claim 1, wherein the end effector comprises one or more hinge elements.

7. The steerable medical instrument of claim 6, wherein the hinge element is a notch.

8. The steerable medical instrument of claim 7, wherein the notch is a T-bar shaped notch.

9. The steerable medical instrument of claim 6, wherein a plurality of hinge elements are disposed along the length of the end effector.

10. The steerable medical instrument of claim 9, wherein the spacing between the hinge elements varies along the length of the end effector.

11. The steerable medical instrument of claim 10, wherein the distance between each hinge element decreases gradually along the length of the end effector from its proximal end to its distal end.

12. The steerable medical instrument of claim 1, wherein one or more hinge elements are disposed on the end effector to enable bending of the end effector in a desired plane of motion.

13. The steerable medical instrument of claim 1, wherein the end effector further comprises an outer sleeve.

14. The steerable medical instrument of claim 1, wherein the end effector further comprises a fluted tip at its distal end.

15. The steerable medical instrument of claim 1, further comprising one or more heat-shrink bands disposed at one or more predetermined locations along the length of the shaft, the end effector, or both.

16. The steerable medical instrument of claim 15, wherein at least one of the one or more heat-shrink bands are capable of being visualized when the instrument is in a patient.

17. The steerable medical instrument of claim 1, wherein the end effector is attached to the distal end of the shaft.

18. The steerable medical instrument of claim 1, wherein the end effector is removably attached to the distal end of the shaft.

19. The steerable medical instrument of claim 1, wherein the end effector is integrally formed in the distal portion of the shaft.

20. The steerable medical instrument of claim 1, wherein the end effector is a composite structure.

21. The steerable medical instrument of claim 1, wherein the end effector comprises at least one of a grasping device, a cutting device, a snare, a specimen retrieval device, or a wound closure device.

22. The steerable medical instrument of claim 21, wherein the wound closure device is a surgical stapler.

23. The steerable medical instrument of claim 1, further comprising one or more balancing lumens in the shaft.

24. The steerable medical instrument of claim 1, further comprising a lumen configured to receive a guide wire.

25. The steerable medical instrument of claim 1, further comprising a lumen configured for the delivery of contrast media.

26. The steerable medical instrument of claim 1, further comprising a lumen configured to receive a guide wire and a separate lumen configured for the delivery of contrast media, wherein the two lumens merge into a single lumen that exits the distal end of the end effector.

27. The steerable medical instrument of claim 26, wherein the end effector has a longitudinal axis and the single lumen exits the end effector at a point that is centered on the longitudinal axis.

28. The steerable medical instrument of claim 1, wherein each of the one or more steering control wires are contained within a separate lumen or channel in the instrument.

29. The steerable medical instrument of claim 1, wherein one or more of the steering control wires run along the outside of the shaft.

30. The steerable medical instrument of claim 1, wherein the shaft further comprises at least one element having a higher modulus than the modulus of the shaft.

31. The steerable medical instrument of claim 30, wherein the at least one element is selected from the group consisting of a wire, fiber, or slug.

32. The steerable medical instrument of claim 31, wherein the at least one element is a metal wire.

33. The steerable medical instrument of claim 31, wherein the at least one element is a fiber comprised of a high modulus polymer or glass.

34. The steerable medical instrument of claim 1, wherein the shaft comprises a mechanically formed, curved section.

35. The steerable medical instrument of claim 1, further comprising a cutting wire, said cutting wire extending distally from the handle through a lumen of the shaft to an exit port, where the cutting wire exits the shaft and runs along the outside of the shaft for a distance, after which the cutting wire enters the shaft at an entry port and is anchored inside the end effector.

36. The steerable medical instrument of claim 1, wherein the shaft further comprises a section located at the distal end of the shaft adapted to be attached to the proximal end of the end effector, wherein the section comprises an internal wire configured to optimize the alignment of the distal end of the end effector.

37. The steerable medical instrument of claim 1, wherein the control handle comprises a gear connected to a steering control wire, and a first position of the control handle actuates the gear to manipulate a steering control wire.

38. The steerable medical instrument of claim 37, wherein a second position of the control handle provides for a neutral position of the gear.

39. The steerable medical instrument of claim 1, wherein the control handle is coated, in whole or in part, to increase traction with the user's fingers or hand.

40. The steerable medical instrument of claim 1, wherein the control handle further comprises a friction pad to lock the steerable surgical instrument in a first position.

41. An end effector for a medical instrument, comprising
a flexible member comprising a proximal end and a distal end, wherein the proximal end is attachable to a medical instrument; and
wherein a material property of the flexible member varies along its length to account for variable bending moments experienced by the flexible member when the end effector is in use in a patient.

42. The end effector of claim 41, wherein the material property of the flexible member is stiffness.

43. The end effector of claim 41, wherein the flexible member comprises one or more hinge elements.

44. The end effector of claim 43, wherein the hinge element is a notch.

45. The end effector of claim 44, wherein the notch is a T-bar shaped notch.

46. The end effector of claim 41, wherein a plurality of hinge elements are disposed along the length of the flexible member.

47. The end effector of claim 46, wherein the spacing between the hinge elements varies along the length of the flexible member.

48. The end effector of claim 47, wherein the flexible member has a proximal end and a distal end, and the distance between each hinge element decreases gradually along the length of the flexible member from its proximal end to its distal end.

49. The end effector of claim 41, wherein one or more hinge elements are disposed on the end effector to enable bending of the end effector in a desired plane of motion.

50. A method for manufacturing a steerable medical instrument, the method comprising:
forming an end effector comprising a distal end, a proximal end, and a longitudinal axis; and
creating a plurality of hinge elements disposed along the longitudinal axis of the end effector.

51. The method of claim 50, further comprising the step of anchoring one or more steering control wires in the end effector.

52. The method of claim 51, further comprising the step of encasing the control wires in a Teflon sleeve to reduce friction.

53. The method of claim 50, further comprising the steps of providing a shaft having a distal and a proximal end, and attaching the proximal end of the end effector to the distal end of the shaft.

54. The method of claim 53, further comprising the step of providing a control handle and attaching the control handle to a proximal end of the shaft.

55. A method of positioning a steerable medical instrument in a patient's body, comprising the steps of:
providing a viewing scope having an instrument channel and an exit port;
providing a steerable medical instrument as defined by claim 1;
navigating the scope through the patient's body and positioning the scope near or adjacent a desired area in the patient's body;
introducing the steerable medical instrument through the instrument channel in the scope and advancing the instrument until the distal end of the instrument protrudes from the exit port; and
steering the distal end of the instrument by tensioning at least one steering control wire.

56. A method of cannulating the Papilla of Vater in a patient, comprising the steps of:
providing a flexible endoscope having an instrument channel and an exit port;
providing a steerable medical instrument as defined by claim 1;
navigating the endoscope through the patient's body and positioning the endoscope so that the exit port is near or adjacent the Papilla of Vater;
introducing the steerable medical instrument through the instrument channel of the endoscope and advancing the instrument until the distal end of the instrument protrudes from the exit port;
further advancing and steering the instrument to enter and cannulate the Papilla, wherein the steering is achieved by tensioning at least one steering control wire.
